(19) United States
(12) Patent Application Publication
(10) Pub. No.: US 2008/0077434 A1
(43) Pub. Date: Mar. 27, 2008

(54) System and Method for Administration of On-Line Healthcare

(76) Inventors: Avner Man, Kibbutz Beit Alfa (IL); Avner Amir, Ramat Ishai (IL)

Correspondence Address: SCHWEITZER CORNMAN GROSS & BONDELL LLP, 292 MADISON AVENUE - 19th FLOOR, NEW YORK, NY, 10017, US

(21) Appl. No.: 11/632,435

(22) PCT Filed: Jul. 15, 2005
(86) PCT No.: PCT/IL05/00758

§ 371 Date: Jan. 12, 2007

(30) Foreign Application Priority Data
Jul. 15, 2004 (IL) 163061

(51) Int. Cl. G06Q 10/00; A61B 5/00

(52) U.S. Cl. 705/2; 600/300

(57) ABSTRACT

The present invention discloses a healthcare administration system useful for the management of anamnesis and medical records, data analysis, guided diagnosis, medical treatment, and clinical investigation. The novel system comprising: a plurality of self-sufficient subsystems adapted to record, store, share, clinically investigate and analyze information by means of a common medical information protocol (CMIP); at least one end-unit device adapted to diagnose and/or treat patients, in communication with a subsystem for controlling, monitoring and recording the treatment process and its outcome by means of a medical protocol; at least one module adapted for a CMIP. The end-unit device is guided by the CMIP so that anamnesis, diagnosis and targeted treatment is dictated, provided, monitored, recorded and/or clinically investigated. The present invention also discloses a guided method for a healthcare administration system, useful for the management of medical records, data analysis, diagnosis, guided treatment and medical investigation by means of the medical system as defined above.

### FILED OF THE INVENTION

The present invention generally relates to a system and to a method for the administration of intercommunicated healthcare. More specifically, the present invention relates to a network-based healthcare administration system, method and means for diagnosis, data analysis and treatment.

### BACKGROUND OF THE INVENTION

Today's healthcare communication systems are hindered by several drawbacks. Medical information is not shared among professionals quickly enough to meet the need to provide rapid and universal development and distribution of medical knowledge. Present medical knowledge databases merely accumulate independent research work. Analyzing this large body of work so as to produce new and efficient medical treatment protocols often requires laborious human work. Such human interface and interpretation is a required link in transforming the stored accumulated clinical and research data into knowledge available to the professional medical community at large. Human labor tends to increase the cost as well as to decrease the reliability of the knowledge distributed. Present medical delivery systems rely on having the patient consult a medical professional for diagnosis and therapy. The professional holds the relevant medical information by virtue of his own education and experience. As a result, significant costs in time and resources are spent on the education and specialization of relatively few professionals who need to respond to the healthcare demands of a large number of patients. This “bottleneck” is a major reason for the ever-increasing cost of medical care in modern developed economies.

A number of inventions have attacked the difficulty of remote care and monitoring (further discussed below) but fail to answer the need to update professionals on the evolution of medical knowledge. This deficiency is due to the lack of a proper “end-unit”—a device to monitor and record medical data during treatment in a simple format. The deficiency is also due to the lack of a data processing module whose function is to generate statistical summaries of accumulated data as well as a module that distributes the results of such data processing

U.S. Pat. No. 6,669,631 to Norris, et al discloses a computing technique employed to mine statistical data bases and patient specific data files contributed from multiple sources, including implantable medical devices (IMD) together with external medical devices and other sources, to formulate patient-specific monitoring, diagnostic, therapeutic and educational information and to deliver the patient-specific diagnostic, therapeutic and educational information to the patient and/or patient health care provider. While such a device provides real time data related to a particular patient, it does not provide treatment data, statistical summaries or updating and distribution mechanisms for the evolution of medical knowledge.

U.S. Pat. No. 6,648,823 to Thompson describes an integrated system and method of bi-directional telecommunication between a web-based expert data center and a medical device, utilizing various types of network platforms and architecture, to inform patients and clinicians, upon connection with the expert data center, about recalls or alerts and recommend courses of action relating to the selection of programmable parameters and the course of treatment/follow-up of an IMD. Such a device provides a remote consultation with a medical expert but does not provide treatment data, statistical summaries or an updating and distribution mechanism for developments in medical knowledge.

Medical communication systems, such as those presented by U.S. Pat. Nos. 6,669,631 and 6,648,823 are designed to perform only with a well-defined hardware-specific medical device. Such systems do not, in general, provide the ability to actually perform variable medical treatment, at the therapist's clinic or patient's home. The scope of such prior art is restricted in its application to implantable medical devices. Such systems do not in any way address the distribution of medical knowledge and improvement of treatment and diagnostic protocols beyond specific applications of the implantable device.

U.S. Pat. No. 6,597,392 to Jenkins et al introduces a computer-implemented diagnostic imaging tool for capturing multi-media data for organization and transmission from a database, and in particular a telemedicine technique using multi-media data capture, storage, and transmission of episodes-of-care for medical consultation.

Other systems presented in the art include various end-units consisting of a multiplicity of separate modules, which collectively perform a useful biomedical purpose; the modules communicating with each other without the use of interconnecting wires. All modules may be intra-corporeal or body mounted extra-corporeal, or some modules may be intra-corporeal with others being body mounted extra-corporeal. Such a system lacks the ability to connect to an on-line main database with a central analysis system used to control large numbers of similar end-units. Furthermore, such a system does not include a common medical protocol for diagnosis, treatment, and sharing of information with other similar systems, or for collecting and analyzing the shared data.

A wide variety of devices found in the market may be used as an end unit for a system for administration of intercommunicated healthcare, but the method of integrating such a system is still missing. Such end units should have a proper energy applicator to perform therapy and/or a sensor or information collector, the latter being used to detect the human body. The energy emitter might be a laser, a light emitting diode an ultrasound wave generating and directing device or any similar device.

A technique known as EAV, i.e., “Electric Acupuncture according to Voll”, after Dr. Reinhold Voll is a well-known method that may be used as an end-unit, utilizing acupuncture treatment. In such a use—an electrical impedance examination of acupuncture points provide the body response data while application of electricity through acupuncture needles is the mode of therapy. The impedance measurement method, also called a “Vega test”, is described in the book “Treatise of Acupuncture” by Charles H. McWilliams, Health Sciences Research, P.O. Box 441242, Miami, Fla., U.S.A. The treatment may also consist of homeopathic or herbal medicines. A wide range of systems utilizing the EAV method is presently available, but there is no such system able to collect the treatment data, analyze it and produce statistical summary data as well as distribute it. Additionally no such system is able to produce treatment and diagnostic protocols, which may be tested, analyzed together with the treatment data records and upgraded by the system. The impedance method is also known to be non-robust and difficult to master due to the difficulty in achieving good and stable electric contact with the patient's skin across a multitude of applications.

In the context of acupuncture end units, with possible improvement on the impedance method, the work by Lazoura et al. (Second International Conference on Bio-electromagnetism, February 1998, Melbourne Australia, pp. 171-172) describes a method of measuring the absorption of light at acupuncture points relative to non-acupuncture points. Although such discrimination was proven, the mechanism used for measurement is bulky and not suitable for clinical work by a therapist or a home-user. Further, it is not suitable as an end unit required under the present invention.

In a report made by MedDynamics Ltd. (Jan. 6, 2004) an experimental setup was built to measure the absorption of light at the neighborhood of such points. The outcome data of these measurements was calculated and presented in two and three dimensionional graphs. A least of acupuncture points was examined (PC4, PC5, PC6, PC7, TW4, TW5, TW6, HT8) compared to their surrounding areas.

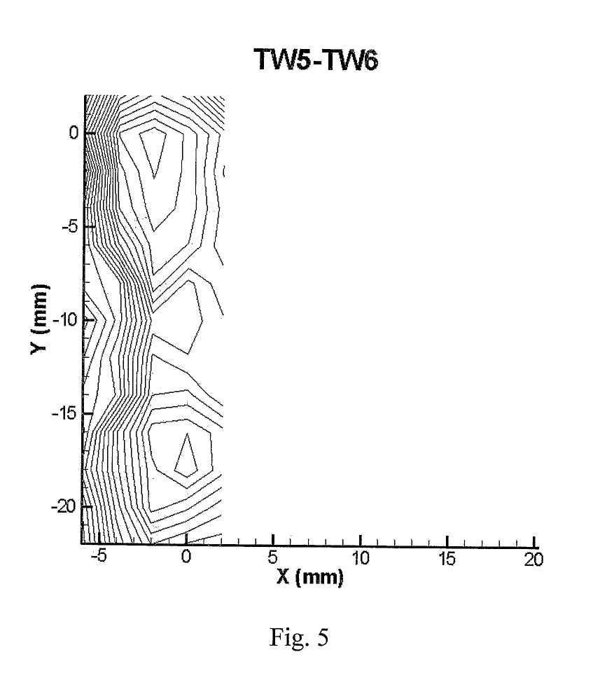

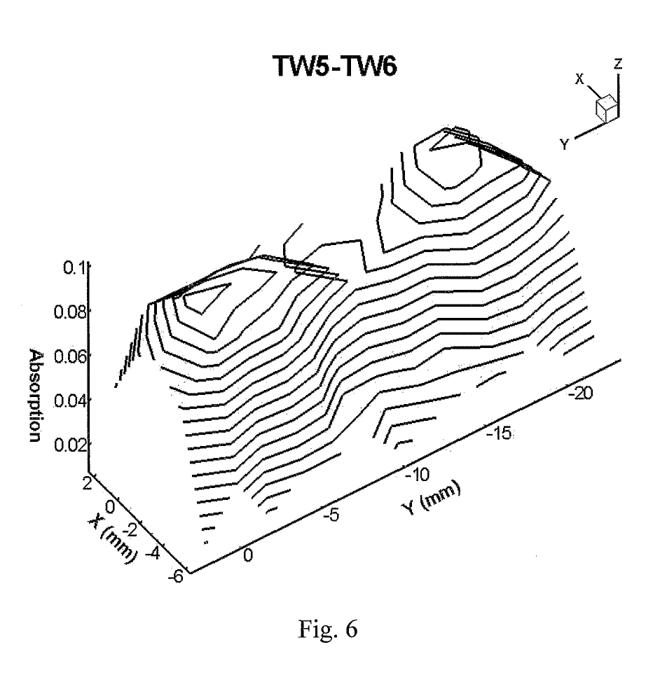

The results presented in the report shows a detailed map of absorption near acupuncture points. The results are in general agreement with earlier results obtained by Lazoura et al. The difference here is that Lazoura et al. investigated only a few points and did not perform extensive mapping as under MedDynamics work. The latter also used a more accurate measurement method of integrating spheres. An example of the graphs is presented at FIG. 5 (TW5 & TW6 in two dimensions) and in FIG. 6 (TW5 & TW6 in three dimensions).

A similar method suitable for a portable device operating on energy emission and detection, said energy in the form of light or ultrasound, is described in a patent application by Amir and Man “A Directed Energy Acupuncture System” submitted simultaneously with the present application.

Cost effective healthcare and therapy systems often require a constantly-updated retrievable database correlating the identity of a specific specialist with an online record describing a specific medical condition. Such knowledge is difficult to obtain in present healthcare administration systems due to the complexity of existing healthcare administration system.

The reliance on and the need to sustain database infrastructure, have generally led modern medicine to emphasize the use of an expensive treatment-oriented approach, rather than developing more cost-effective modalities emphasizing a preventive approach.

The relatively low cost of information storage and exchange as made available by data processing and storage technology can potentially alleviate some of the above difficulties if adopted by the various components (systems, professionals and patients) of a healthcare administration system. However, due to the lack of a general integrative system, such as needed to make optimal use of such facilities, with a method specifically aimed at distributing medical knowledge and care has not been invented to-date. A major roadblock for such a system are the lack of integration of specific appropriate treatment, diagnostic and care-delivery elements all working under a common medical platform.

### SUMMARY OF THE INVENTION

It is thus one object of the present invention to provide a healthcare administration system useful for the management of medical records, data analysis, diagnosis, guided treatment and clinical investigation. This novel system comprises a plurality of stand-alone subsystems adapted to share and process information by means of a common medical protocol; at least one end-unit device adapted to diagnose and/or treat patients, in communication with a subsystem for controlling, monitoring and recording the treatment process and its outcome by means of a medical protocol; and at least one module adapted to an information protocol that we shall refer to as a common medical information protocol (CMIP), wherein said end-unit device is a guided by said CMIP so that anamnesis, diagnosis and targeted treatment is, dictated, provided monitored, recorded and/or clinically investigated. In the present disclosure the terms “system” and “subsystem” may be used interchangeably as the character of the invention permits one component to serve as a fully self-sufficient system in one configuration while it may be interconnected as a subsystem in a different configuration.

The use of end unit devices allows the application of control and monitoring mechanisms. The definition of a quantitative set of control and monitoring parameters forms a medical treatment protocol. The set of parameters used in the application of treatment may include control parameters such as intensity, duration, repetition, wavelength and location of treatment. The end unit combines the capabilities of an energy applicator, applying the treatment to the human body, and a monitoring element. The monitoring device, such as a light sensor, a CCD camera or an ultrasound-imaging device, permits recording of the effect of the energy on the patient and the production of a digital record of the treatment and its effect.

The use of protocols and associated records also permits communication of results to remote experts. Medical data of the type described here require the use of a special data structure. Hereafter we shall refer to such a structure as a “Common Medical Information Protocol” (CMIP). Such protocol is essentially a set of data items necessarily part of such a system. Such data items include treatment, diagnosis, communication and other items. The aforesaid CMIP module is preferably selected from the group of: patient data archiving module; practitioner data archiving module; anamnesis data module; diagnostic protocols data module, diagnosis registration data module; treatment protocols data module; clinical investigation management module; medical knowledge data module; information security data module; registry data module; controlling, monitoring and recording module, data retrieving module or any combination thereof.

It is in the scope of the present invention wherein the end-unit device is an energy emitting device such as a laser or an ultrasound emitter together with a sensing device to measure the effect of the irradiated energy on the patient's body, such sensor being for example an CCD image sensor device comprised of an applicator and sensor applying a medical treatment at a treatment point. A similar device may also consist of an ultrasound emitter and an ultrasound-imaging sensor. Both such end units are described in a patent application by Amir and Man “A Directed Energy Acupuncture System” submitted simultaneously with the present application.

The aforementioned system may comprise at least one sensor and one transmitter; said sensor is adapted for detecting predetermined physical, biological or chemical parameters; said transmitter is adapted to transmit said detected parameters so that remote anamnesis, diagnosis and targeted treatment is dictated, provided monitored, and/or recorded. It may additionally comprise software adapted for collecting the transmitted data and analyzing said data so as to produce a medical diagnosis. It may additionally or alternatively comprises a unit for collecting the therapy data for future review and/or statistic analysis and/or for clinical investigation.

It is also in the scope of the present invention wherein the system defined above is adapted to inter-communicate data, and especially data organized in a CMIP structure, and whose modules are selected from the aforementioned group of modules.

It is also in the scope of the present invention wherein the end unit is comprised of at least two compatible parts: end unit hardware, and a software driver; said end unit hardware is designated as a device providing the analysis and/or treatment and said end unit software is adapted to facilitate interfacing of said end-unit with user's computer, operating system or other software on it.

The disclosed system may be embodied in the form of a home use system, a professional system, an enterprise system or a large enterprise system. Such systems form a hierarchy where any of the above may be networked to the other systems following in the sequence. The network enables the systems to exchange information. Such information includes the exchange of treatment protocols, summary data and other information. The transfer of information contributes to accumulation of summary data and the evolution of medical knowledge. The evolved medical knowledge results in the generation of better treatment procedures. Due to the fact that network connection permits rapid exchange of information, a rapid evolution of medical knowledge is made possible.

In one embodiment the system, or an element of such, may be useful as a home-use system for patients; comprising a personal computer; an end unit, an on-line connection and management software, so that non-professional users are permitted and guided to analyze and/or treat predetermined medical problems at home. A home use system may also be used as a self-administered mobile medical system, using a palm computer or a cellular portable phone, connected to an end unit or embedded in it.

In another embodiment the system or an element of it may be used by a medical professional, hereafter termed a “professional system”. Such embodiment comprises a personal computer; an end unit, an on-line connection and management software adapted for use by medical practitioners;

In another embodiment the system or an element of it is useful as a healthcare provider (hereafter termed “enterprise”) system utilizable professionally. A clinic enterprise system may be an enterprise system adapted to be used by medical clinics; comprising a server computer; at least one end unit; an on-line connection to the sub-systems; an on-line connection to the main system, and management software. A healthcare provider enterprise system may be an enterprise system alternatively adapted to be used by healthcare providers comprising a server computer; at least one healthcare provider subsystem adopted for use in a clinic; an on-line connection to the sub-systems; an on-line connection to the main large enterprise system, and management software. A large enterprise system may also be an enterprise system adapted to be used by large organizations such as World Health Organization or a country's ministry of health preferably comprising a server computer; at least one end unit; an on-line connection to the sub-systems; an on-line connection to the main system, and management software.

It is another object of the present invention to provide a guided method for a healthcare administration system useful for the management of medical records, data analysis, diagnosis and guided treatment by means of the medical system as defined in claim 1 or in any of its dependent claims. This method may comprise the steps of interconnecting a plurality of self-sufficient subsystems adapted to share information by means of a common medical protocol and at least one end-unit device adapted to diagnose and/or treat patients, in communication with a subsystem for controlling, monitoring and recording the treatment process and its outcome by means of CMIP; wherein said end-unit device is a guided by said CMIP so that anamnesis and targeted treatment is dictated, provided monitored, recorded and/or clinically investigated.

It is in the scope of the present invention wherein the aforesaid method additionally comprises the step of communicating with a database, so that data archiving and/or data retrieving can be carried out. Said method may additionally comprise the step of collecting the transmitted analysis data and analyzing said data so as to produce a medical diagnosis in a remote location; and/or the step of collecting the therapy data for future review, for clinical investigation and/or for statistical analysis. This method may alternatively usefully be implemented by a healthcare provider (i.e., enterprise) system, comprising a server computer; at least one end unit; an on-line connection to the sub-systems; an on-line connection to the main system, and administrating software.

### BRIEF DESCRIPTION OF THE INVENTION

In order to understand the invention and to see how it may be implemented in practice, a preferred embodiment will now be described, by way of non-limiting example only, with reference to the accompanying drawing, in which

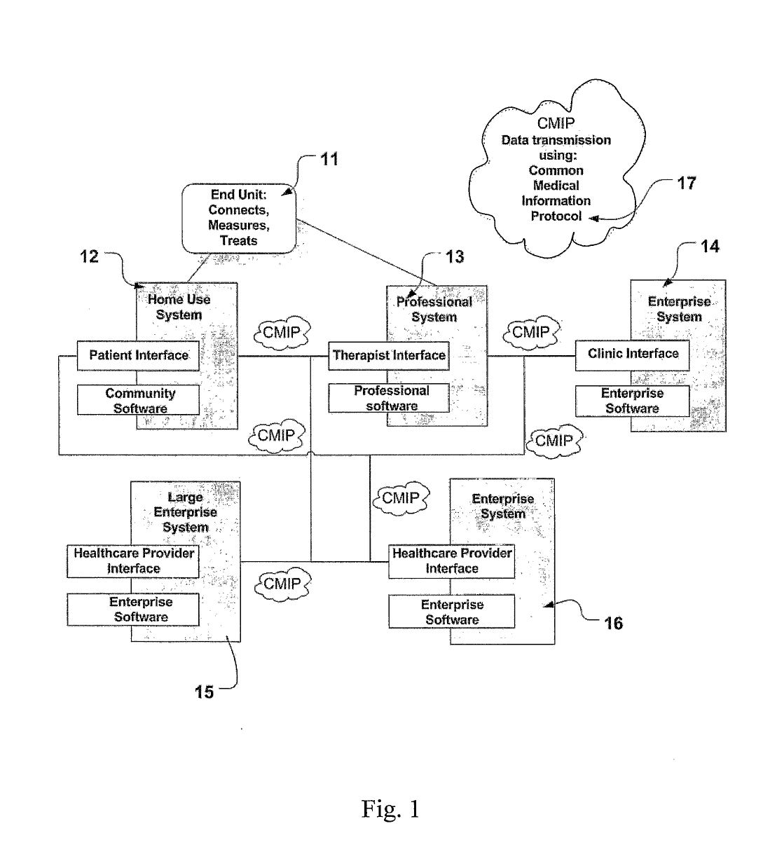

FIG. 1 schematically presents a network collection of systems including home, professional, enterprise and large enterprise systems. Each system may also be considered as a subsystem, which together with other subsystems forms an integrated system. The networked system exchanges information using a CMIP;

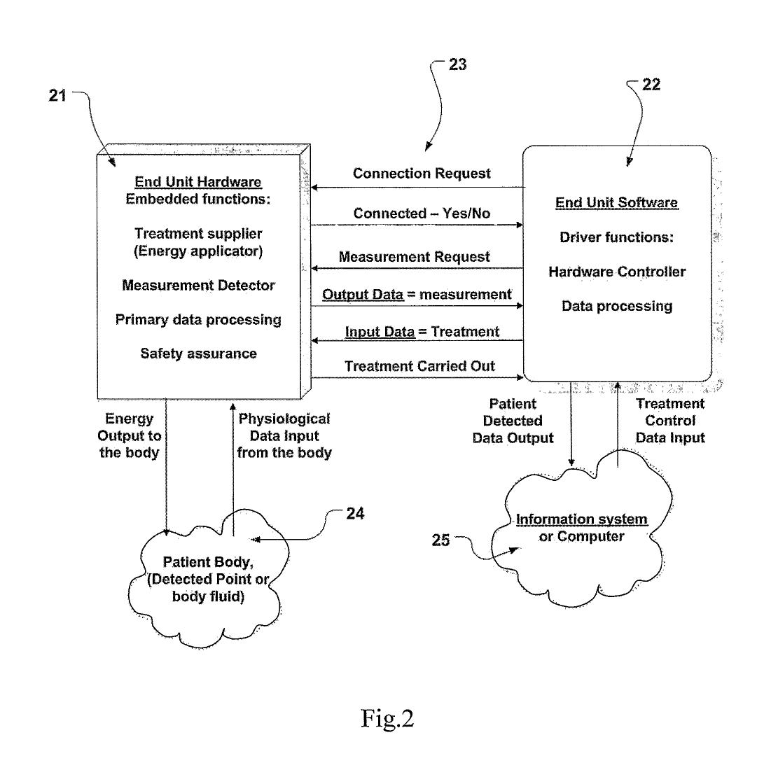

FIG. 2 schematically presents an end-unit model. The figure illustrates various components of the information being transmitted and processed during end-unit operation;

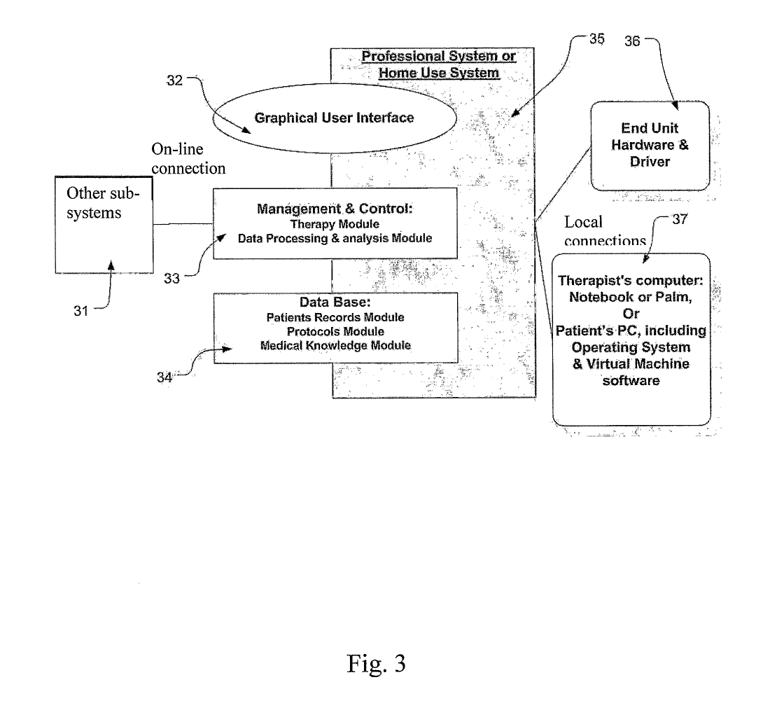

FIG. 3 schematically presents professional and a home use systems;

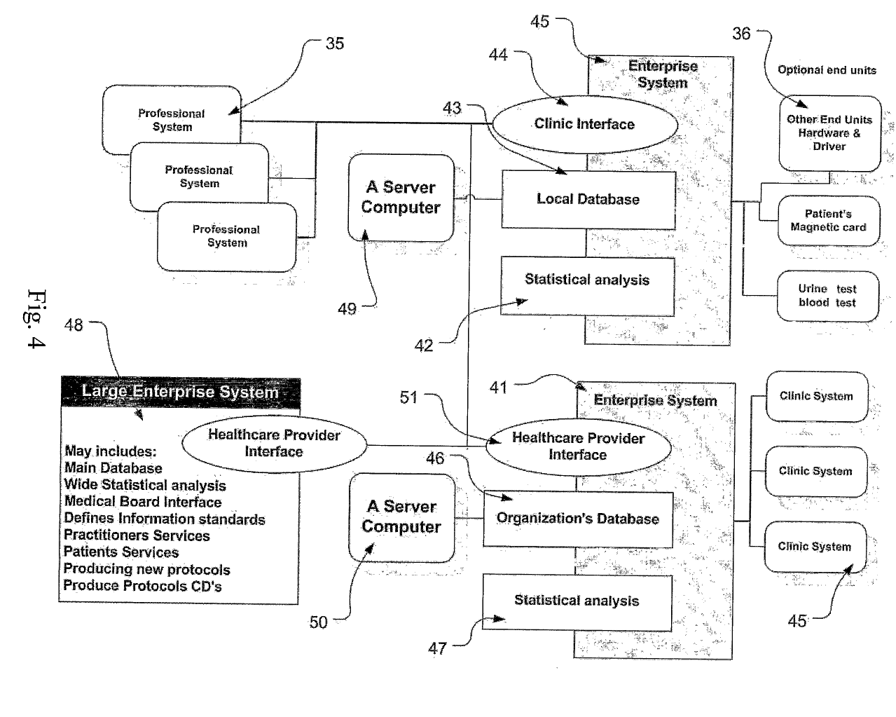

FIG. 4 shows a large enterprise system model; for example, healthcare provider system networked to a net of enterprise subsystems used in clinics;

FIG. 5 presents a two-dimensional graph of results displaying a map of light absorption at acupuncture points (TW5 & TW6); and,

FIG. 6 presents a three-dimensional graph of results displaying a map of light absorption at acupuncture points (TW5 & TW6).

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a network-based healthcare-administration system.

The term “administration” hereinafter applies to the method and process of administering and providing healthcare as well as medical knowledge.

The novel method and system according to the present invention automatically collect information on-line from many healthcare professionals engaged in treating patients. The collected information includes, but is not limited to, methods of diagnosis and treatment, and records of patient medical data. The collected information is accumulated to form a main database of medical knowledge. Data processing on the main database is performed continuously so as to generate a summary of medical information. Data processing can also be performed at any of the subsystems, to free the main system from overload, and collected at a system higher in the hierarchy. Such summaries and/or the outcome of the analysis thereof are collected and distributed amongst professionals, thereby contributing to the evolution of medical knowledge. The medical data summaries are sent to the professionals connected to the system, by means of CMIP. The subsystems, on the other hand, collect and analyze the treatment data to form suggested diagnostic and treatment protocols, which in turn are sent to the main system, are clinically investigated, and/or statistically analyzed, and depending on the investigation results are registered at the main system as CMIP. Such a method enables the protocols to be updated automatically on a regular basis, such that the patients are able to obtain an updated therapy from a system that is being improved on a continual basis. The system can also be used to provide the relevant medical information concerning the patient's health to both the patient and the therapist.

The system disclosed here uses currently available information technologies together with special end-unit devices capable of collecting reliable data from the human body. Such data is organized in a common data structure that allows automated processing and effective application of diagnosis, treatment and clinical investigation.

The term “administration” refers hereinafter to a method and process of administering and providing healthcare.

Reference is made now to FIG. 1, presenting a global system model, which interconnects separate system elements to form a united integrated system. Each system elemental; i.e. end unit (11), home use system (12), professional system (13), enterprise clinic system (14), enterprise healthcare provider system (16) and enterprise large system (15), of the global system is adapted to communicate with other elements, so as to permit sharing of medical information and summary data. The information thus communicated is formatted by means of a Common Medical Information Protocol (CMIP) (17). The CMIP may include records of information recognized by all elements such as: patient data archiving module; practitioner data archiving module; anamnesis data module; diagnosis protocols data module, diagnosis registration data module; treatment protocols data module; clinical investigation management module; medical knowledge data module; information security data module; registry data module; controlling, monitoring and recording module, data retrieving module. The CMIP may further include a header, which summarizes the information included in the information sent, for example: number of packages sent, their size, and any other relevant information.

The terms ‘patient data archiving module’ and ‘practitioner data archiving module’ refer hereinafter to patient's personal details, such as first name, family name, home address, country, home phone, mobile phone, work phone, I.D. number, date of birth, place of birth, hour of birth, marital status, passport photo, etc. The terms may also refer to professional details such as: medical license number, country of license, date of license, institute licensed, publications, etc.

The term ‘diagnosis protocols data module’ refers hereinafter to any method and diagnostic procedure according to chief complaint and including other symptoms. For example, headache anamnesis protocol, constipation anamnesis protocol, etc. The term may also relate to a method and procedure for determination of diagnosis by a standard examination, such as hair examination anamnesis protocol, urine examination anamnesis protocol, etc.

The term ‘diagnosis registration data module’ refers hereinafter to a least of registered data, documenting a wide spectrum of existing diagnoses. It is related to specific diagnoses registered on the system for example: “Headache type 125”, “Hard dark stools constipation type 15”, etc. An example of such least is the ICD10 code, available from the World Health Organization.

The term “treatment protocols data module’ refers hereinafter to any method and tools of treatment according to specific diagnosis, such as “Headache type 125”, “hard dark stools constipation type 15”, etc. It may also relate to any method and tools of treatment according to targeted achievement, such as “strengthening digestion” protocol, or “improving sporting abilities type 11” protocol, etc. The applicative content of the treatment protocol may be further illustrated by referring to one of the examples of the end-units described below.

For a laser type device the treatment application protocol may take the form of one or more of the following examples:
1. Name of Protocol: Strengthening Digestion; Device: Laser; Mode: Pulsed Wavelength: 808 nanometer; Average power: 150 milliwatt; Duty-cycle: 75%; Deposited energy: 3 Joule; Treatment point: Acupuncture—Sp6, St36, Pc6, Cv12; Weekly: 3 times; Total applications: 12 sessions etc.
2. Name of Protocol: Strengthening Immune System; Device: Laser; Mode: Pulsed Wavelength: 808 nanometer; Average power: 120 milliwatt; Duty-cycle: 50%, Deposited energy: 2 Joule; Treatment point: Acupuncture—Sp6, St36, Lu7, CV6; Weekly: 3 times; Total applications: 12 sessions etc.

For an ultrasound type device the treatment application protocol may take the form of one or more of the following examples:
3. Name of Protocol: Tennis Elbow Pain; Device: Ultrasound; Mode: Pulsed frequency: 30 KHz; Average power: 5 Watt; Duty-cycle: 50%; Deposited energy: 30 Joule; Treatment point: Acupuncture—LI11, LI12; Weekly: 3 times; Total applications: 9 sessions etc.

The term ‘medical knowledge data module’ refers hereinafter to the summary of information obtained from the data analysis of the gathered information, e.g., data relevant for informing patients or professional about healthy life style, physical activity, nutrition etc. Such information may further assist the patients in improving their health and preventing disease. Medical knowledge may also include specific medical information about specific illness from a physiological or psychological point of view. The term may also include medical findings for practitioners, outcomes of clinical investigations, medical articles and examples of imaging in such cases.

The term ‘Information Security Data Module’ refers hereinafter to security data records that may include the preferred security level of the user, so as to restrict access and preserve confidentiality. For example, a patient may want to send medical information to his health practitioner, but does not want his private details to be delivered to other practitioners, he is willing to allow the practitioner to send the information for consultation without personal details. While sending the CMIP to the practitioner the patient's system will add a Security Data Record that informs the practitioner how to deal with the information.

The solution in such a case might be to open a “case history” profile on the practitioner's system and use it as “patient details for consultation”, and thus respect the patient's privacy. In a “case history” file, the private details will be replaced with a “private patient's number” on the “number of practitioner system”, thus ensuring that the system records who owns the information, which is important for statistic analysis.

The term ‘Registry Data Module’ refers to a record or module that restricts the sharing of certain data to within a group of registered subsystems only. One purpose of the registry data record is to permit the existence of a closed system and separately of an open system. The closed system is designed to ensure restricted access to certain items of data amongst its members.

The terms “open system” and “closed system” may be generally characterized in a non-limiting manner as systems wherein open system is a free system on the private market, such as a patient consulting with a private practitioner on-line, without a meeting and without being registered on the practitioner's private system. In such a case there is no real obligation between them because formally the patient is “anonymous”; while a closed system may be operated, for examples by a healthcare provider, who keeps a record of each of its patient who are registered on one of its own subsystems. A closed system can rely on the statistical information it owns and create its own data summaries for internal use, not to be shared or partially shared by external sub-systems. In a closed system the medical history of the patient, for example, is preserved and may be reached all over the system.

A closed system can perform a reliable clinical investigation on its collected information with the aim of developing reliable diagnostic and treatment protocols. An open system, in contrast, cannot perform reliable clinical investigation because, for example, many different “anonymous users” might in fact be the same person, meaning that any analysis or conclusion based on the information collected by an open system is unreliable.

Two “closed systems” may agree to share statistical information. In this case they have two options (i) Set up a united registry and become one closed system with a united integrated system, and an integrated database. In this way they may share individual patient data or “raw” data, that is the pure data items in the form they are gathered (not only summaries), which enable the united system to analyze a wider range of data; or (ii) Share only summary information and derived knowledge utilizing common trust as to the reliability of each other's information, but without integrating their registry or databases. The two systems can then compare summaries but they are unable to combine all raw data, and they will not have an outcome of common data analysis. In both such cases it is important for both systems to recognize and support the same CMIP, which allows them to share forms of medical data.

It is in the scope of the present invention wherein several variations are possible for achieving therapy. For example, a patient undergoing a therapy may be receiving it by means of an end-unit that is connected to a therapist's computer. The patient may also be connected directly to the healthcare provider sub-system in a clinic. The patient may also be getting on-line medical consultation and/or treatment using a home use subsystem. The patient may connect as a subsystem using his personal computer or using a mobile device specially designed for that purpose or included with a cell phone.

A therapist might administer treatment directly to the patient at the clinic, or by on-line connection through the patient's home system, as a consultant. A therapist can also work in a private clinic or at a private shared clinic or through a health provider's clinic. A healthcare provider can activate a net of information and treatment accessible directly to the patient, or through a network of clinics. The health provider may want to centralize the information as a main system, or share it with other healthcare providers for mutual benefit through a separate shared central system.

Reference is made now to FIG. 2, presenting a schematic diagram of end unit components. The common requirements of an end-unit are as follows: (i) Provide a method of use and ability to reliably measure the relevant physiological and if necessary anatomical information; (ii) A method of applying treatment to the human body.

The term ‘end units’ refers hereinafter to medical devices able inter alia to interface to the human body (24) in a manner permitting them to measure the body's physiological and possibly anatomical information reliably as their data input.

The method of application of medical treatment by the end-unit (21) utilizes various forms of energy that can be applied to the human body, such as heat, light, sound, pressure, magnetic or electric field, or chemical agents applied to the body. Moreover, the information collected from the patient during the therapy process is entered into standardized data files, which permit computerized processing.

It is in the scope of the present invention wherein aforesaid ‘End Unit’ comprises at least two compatible parts: end unit hardware (21), and end unit software (22).

The end unit hardware (21) is a designated device providing the treatment. Hence, it may be interfacing to, may be probing and /or treating the human body (24). This device may include hardware and embedded functions to control the energy supplier and the detector, and may include embedded software for fast primary data processing of the information it receives from the body. The end unit software (22) is the part of software adapted to facilitate interfacing (23) of said end-unit with user's computer operating system and the software on it (25).

End-units of various types are possible. Each one of the following embodiments is capable of operating as an individual system (only connected to its own driving computer (25)) or as a subsystem of a larger integrated system.

An example is a laser acupuncture end-unit system, invented and disclosed under a separate patent application by Amir and Man “A Directed Energy Acupuncture System” submitted simultaneously with the present application.

Such a laser acupuncture system is a self-administrated medical device for applying laser acupuncture anamnesis and treatment. The system's end unit includes a laser source and a detector for measuring the photo-absorption of an acupuncture point. The unit is capable of locating the acupuncture point, measuring its functionality, and at a later stage applying laser treatment to the point.

The system uses its end unit for a diagnostic procedure including locating and examining a series of acupoints and analyzing the information according to a protocol. The diagnosis is received on the system, which further uses the end unit to perform treatment according to a computerized protocol. The system records the diagnosis and treatment information for further analysis; enable the medical professional to manage clinical investigation during daily practice of medical treatment.

Reference is now made to FIGS. 5 and 6. These figures demonstrate the capability of an end unit to monitor light energy absorbed in the body in the course of treatment and also detect acupuncture points. FIG. 5 depicts a two-dimensional absorption map obtained when a light sensor and source is scanned over an area in the vicinity of two acupuncture points (TW5 and TW6). Fig, 6 displays a three-dimensional rendering of the same measurement. The maps are values of the absorption measured while the peaks in the maps indicate the position of the preferred treatment points according to recognized physiological principles.

Reference is made now to FIG. 3, schematically presenting professional or home use systems (35), comprising different levels of diagnostic capabilities and providing protocols for diagnostic and treatment procedures. Such a system (35) is connected to at least one end unit (36), using computer (37), software for operating the system using graphical user interface (32), management software for medical data processing (33) and a database (34). By connecting to a main system (31), which gathers the data arriving from many similar subsystems, the integrated main system performs wide ranging statistical analyses, thus enabling evolution of medical knowledge. The integrated main system sends this knowledge to the subsystems.

Previously referred to end-units have in common characteristics which allow the system to interact with the human body in terms of input/output and by doing so the information gathered by the system can be made to contribute to medical knowledge that is shared amongst professionals and improves actual treatment.

An analogy that further illustrates the relationship of existing devices and their enhanced value when used as end-units may be drawn from the world of digital music. To generate music one may use a microphone and speaker, combined to a sound card on a computer as an input/output device of sound. In generating the composer may be influenced by other music he might have heard, but with no physical means to combine it the introduction of such music into his own creation is only subjective in manner. Also, without a network—the sharing of music is possible only by delivering actual physical recording in the form of disks. In contrast—when using computer systems and networks the composer may record his own generated voice and sounds and combine it directly with other people's voices and sounds delivered to him. He can then deliver the information as digital sound formatted by a standard file format over the Internet.

In a similar manner the present invention allows such an exchange in the field of medicine: a practitioner can record medical information with a properly interfacing end-unit, format it as a standard digital file, exchange it with other users. This exchange allows creation of summary data thereby contributing to general medical knowledge that in turn permits individual professionals to provide better treatment to their patients.

The term “Home Use System” refers to a subsystem especially adapted for non-professional users, by means of safety and treatment abilities, which allow the patient to treat medical problems at home when these problems are minimal. By doing so the system provides more efficient medicine, with a preventive aspect, and encourages a cost effective global medical system.

Using the home use system the patient has the ability to connect to a main database to obtain medical information and so improve self-care opportunities.

Such a home use system (35) may include a personal computer; an end-unit; an on-line connection; management software etc as defined below:

A personal computer (37) such as IBM® ThinkCenter, with an operating system such as one of Microsoft® Windows® products, or a free-to-use Linux® distribution, might use third party software such as SUN® Javaplatform® or Microsoft(® .NET® platform and CLR® software.

An end-unit (36) as described above, might be connected to the system wirelessly with, for example, Bluetooth technology. or possess an on-line connection to the main system (31), such as asymmetric digital subscriber line (ADSL) fast cable connection, or a cellular connection such as CDMA technology.

The management software in this system is for home use, for example community edition software, that includes modules such as GUI (32), management & control (33)(therapy module, data processing & analysis module), local database (34)(patients records module, protocols module, medical knowledge module)

The database is usable, for example, by a family, or might be for a specific medical problem providing the treatment and care for the patient who own it. The medical knowledge obtainable online is limited to home use ability and safety considerations. The protocols module includes a diagnostic module, and a treatment module. Both types of protocols may be updated on-line as needed.

The term ‘Professional System’ refers hereinafter to a subsystem adapted for use by medical practitioners and may include: a personal computer (37) such as IBM® ThinkCenter with an operating system such as one of Microsoft® Windows® products, or a free-to-use Linux® distribution. It might use third party software such as SUN® Javaplatform®, or Microsoft® .NET® platform and CLR® software.

An end-unit (36) as described above, might be connected to the system wirelessly with, for example, Bluetooth technology, or possess, an on-line connection to the main system (31), such as ADSL fast cable connection, or a cellular connection such as CDMA technology.

Management software, for example professional edition software, includes modules such as GUI (32), management & control (33) (therapy module, data processing & analysis module), local database (34)(patients records module, protocols module, medical knowledge module). The management software in this system is for professional use, so that the database is large enough to be capable of holding the records of the practitioner's patients, and might be adapted to the specific medical specialty of the practitioner. The medical knowledge available online is extended for professional needs. The protocols module includes a diagnostic module, and a treatment module. Both types of protocols might be updated on-line as needed.

Reference is made now to FIG. 4, schematically presenting an enterprise system model; i.e., healthcare provider system, which is connected to a larger main system. Professional systems (35) are utilizable in professional clinics (45), while a network of clinics (45) is connected to the healthcare provider's main system (41). Additional end units (46) may be connected at the clinic system, for clinical or administrative needs (for example blood testing device or magnetic card reader for opening a “patient visit” at the clinic). Each subsystem collects data from its own subsystems to a local database (43, 46). Each subsystem is responsible for its own statistical analysis (42, 47), which is performed continuously and simultaneously as part of the system. A security standard (a part of CMIP) is able to keep the patients private details inside a subsystem, while sharing only statistical information with other systems.

There are at least three types of enterprise systems, one for use in a clinic (45), one for use by healthcare providers (41), and a larger enterprise system (48) for use by government regulatory bodies or insurance companies.

The term Enterprise System (45) using a Clinic Interface (44) refers hereinafter to a system adapted to be used by medical clinics and may include: a server computer (49) such as IBM® eServer® BladeCenter, with an operating system such as one of Microsoft® Windows® products, or a free-to-use Linux® distribution. It might use third party software such as SUN® Javaplatform® or Microsoft® .NET® platform and CLR® software.

An end unit (36) as described above, that might be connected to the system wirelessly with, for example, Bluetooth technology, or possess an on-line connection to the sub-system (35) (Professional Systems, etc), e.g., LAN cables connection, or a wireless LAN, or an on-line connection to the main system (41), for example, ADSL fast cable connection, or a cellular connection such as CDMA technology.

A clinic interface (44), for example enterprise edition software, may include modules such as GUI, management & control (therapy module, data processing & analysis module), local database (43)(patients records module, protocols module, medical knowledge module).

The clinic interface in this system (44) might be software for use in a clinic, meaning that the database (43) is large enough to be capable of holding the records of all the patients served by the clinic, and might be adaptable for the medical specializations of the practitioners in the clinic. The analysis module (42) analyzes data from all subsystems, to allow administration and upgrading of clinical protocols. The medical knowledge and protocols it obtains online from-the larger system are extendable for enterprise needs.

The Enterprise System (41) using a Healthcare Provider interface (51) refers hereinafter to an enterprise system adapted for use by healthcare providers. It may include:

A server computer (50) such as IBM(® eServer) BladeCenter, with an operating system such as one of Microsoft® Windows® products, or a free-to-use Linux® distribution. It might use a third party software such as SUN® Javaplatform® or Microsoft® .NET® platform and CLR® software, or posses an on-line connection to the sub-systems (45) (Clinic Enterprise Systems, etc), e.g., LAN cable connection, or ADSL; an on-line connection to the main system, and management software,

A healthcare provider interface (51), for example enterprise edition software, includes modules such as GUI, management & control (therapy module, data processing & analysis module), local database (46)(patients records module, protocols module, medical knowledge module).

The Healthcare Provider Interface (51) might be enterprise software adapted for use in an organization such as a healthcare provider, meaning that the database (46) is large enough to be capable of holding the records of all the patients served by the organization and might be adaptable for the medical needs and abilities of the clinics and professionals in the healthcare provider organization. The analysis module (47) analyzes data from all subsystems; i.e. from all clinics, to allow administration and upgrading of clinical protocols throughout the organization. The medical knowledge and protocols obtained online from the larger system are extendable for enterprise needs. Statistical analysis allows the enterprise system to perform routine clinical investigations during the daily medical practice of the system, to upgrade and reaffirm its medical protocols (CMIP) in use.

The term ‘large enterprise system’ (48) refers hereinafter to a government regulatory body or insurance company, or a very large private organization and may include: A server computer; an on-line connection to the sub-systems (41) (Healthcare Provider Enterprise Systems, etc); an on-line connection to the main system, for example, ADSL (Asymmetric Digital Subscriber Line) fast cable connection, or a cellular connection such as CDMA Technology and management software. Such a large enterprise system may manage its own collected data, analyze its own statistics and determine its own protocols for diagnosis and treatment, to be used by its subsystems as CMIP.

1. A healthcare administration system useful for the management of anamnesis and medical records, data analysis, guided diagnosis, medical treatment, and clinical investigation; said system comprising:
a. a plurality of self-sufficient subsystems adapted to record, store, share, clinically investigate and analyze information by means of a common medical information protocol (CMIP);
b. at least one end-unit device adapted to diagnose and/or treat patients, in communication with a subsystem for controlling, monitoring and recording the treatment process and its outcome by means of a medical protocol;
c. at least one module adapted for a common medical information protocol (CMIP);
wherein said end-unit device is guided by said CMIP so that anamnesis, diagnosis and targeted treatment is dictated, provided, monitored, recorded and/or clinically investigated.

2. The system according to claim 1, wherein the CMIP's module is selected from the group of patient data archiving module; practitioner data archiving module; anamnesis data module; diagnosis protocols data module, diagnosis registration data module; treatment protocols data module; clinical investigation management module; medical knowledge data module; information security data module; registry data module; controlling, monitoring and recording module, data retrieving module or any combination thereof.

3. The system according to claim 2, wherein the end-unit device is an assembly for directing energy for performing treatment on a patient, said assembly comprising means for emitting energy to be directed toward the patient's body, and communication means.

4. The system according to claim 2, wherein the end-unit device also includes a device monitoring the energy absorbed in the body; said energy includes forms of light, electromagnetic wave, sound, ultrasound, pressure wave and other forms of radiation.

5. The system according to claim 2, wherein the end-unit device is an energy emitting device comprising an applicator and sensor applying a medical treatment at a treatment point; said energy includes forms of light, electromagnetic wave, sound, ultrasound, pressure wave and other forms of radiation.

6. The system according to claim 2, additionally comprising at least one sensor and one transmitter; said sensor is adapted to detect predetermined physical, biological or chemical parameters; said transmitter is adapted to transmit a energy form for treatment and/or diagnosis, characterized by any of the said detected parameters, so that remote anamnesis and targeted treatment is dictated, provided, monitored, and/or recorded; said energy includes forms of light, electromagnetic wave, sound, ultrasound, pressure wave and other forms of radiation.

7. The system according to claim 2, additionally comprising software adapted to collect the transmitted data and analyze said data so as to help the medical professional in producing a medical diagnosis.

8. The system according to claim 2, additionally comprising software adapted to use a treatment protocol for performing a treatment according to a defined diagnosis.

9. The system according to claim 2 additionally comprising a unit for collecting the therapy data for future review and/or statistical analysis.

10. The system according to claim 2, additionally comprising software to perform statistical analysis for improving the treatment and diagnostic protocol.

11. The system according to claim 2, additionally comprising software for analyzing the collected data according to generally accepted clinical investigation so as to enhance the reliability of treatment and diagnostic protocols.

12. The system according to claim 2, adapted to intercommunicate data selected from the group of: patient data archiving record; practitioner data archiving record; anamnesis data record; diagnosis protocols data record, diagnosis registration data record; treatment protocols data record; clinical investigation management record; medical knowledge data record; information security data record; registry data record; controlling, monitoring and recording record, data retrieving record or any combination thereof.

13. The system according to claim 2, adapted to intercommunicate in either an open system or a closed system or any combination thereof.

14. The system according to claim 2 wherein the end unit comprises at least two compatible parts: end unit hardware, and end unit software; said end unit hardware is a designated device providing the analysis and/or treatment and said end unit software is adapted to facilitate interfacing of said end-unit with user's computer, its operating system and/or other software on it.

15. The system according to claim 2, wherein the subsystems are especially useful as a home use system; comprising a personal computer; an end unit, an on-line connection and management software, so that non-professional users are permitted and guided to analyze and/or treat predetermined medical problems at home.

16. The system according to claim 2, wherein the subsystems are especially useful as mobile self use systems; with or without a palm computer; with or without a cell phone; an end unit, an on-line connection and a management software, so that non-professional users are permitted and guided to analyze and/or treat predetermined medical problems in any location.

17. The system according to claim 2, especially useful as a professional system; comprising a personal computer; an end unit, an on-line connection and management software adapted for use by medical practitioners.

18. The system according to claim 2 or any of its dependent claims, especially useful as a healthcare provider (i.e., Enterprise) system utilizable professionally.

19. A clinic enterprise system according to claim 18, adapted to be used by medical clinics; comprising a server computer; at least one end unit; an on-line connection to the sub-systems; an on-line connection to the main system, and management software.

20. A healthcare provider enterprise system according to claim 18, adapted to be used by healthcare providers, comprising a server computer; at least one end unit; an on-line connection to the sub-systems; an on-line connection to the main system, and management software.

21. A large enterprise system according to claim 18 comprising a server computer; at least one end unit; an on-line connection to the sub-systems; an on-line connection to the main system, and management software.

22. A guided method for a healthcare administration system, useful for the management of medical records, data analysis, diagnosis, guided treatment and medical investigation by means of the medical system as defined in claim 1 or in any of its dependent claims.

23. The method according to claim 22, comprising the steps of interconnecting a plurality of self-sufficient subsystems adapted to share information by means of a common medical protocol and at least one end-unit device adapted to diagnose and/or treat patients, in communication with a subsystem for controlling, monitoring and recording the treatment process and its outcome by means of of CMIP; wherein said end-unit device is guided by said CMIP so that anamnesis, diagnosis and targeted treatment is dictated, provided, monitored, and/or recorded.

24. The method according to claim 22, additionally comprising the steps of communicating with a database, so that data archiving and/or data retrieving is applicable.

25. The method according to claim 22, additionally comprising the step of collecting the transmitted analysis data and analyzing said data so as to produce a medical diagnosis in a remote location.

26. The method according to claim 22, additionally comprising the step of collecting the transmitted analysis data and analyzing said data so as to produce a medical diagnosis in a remote location, with the aid of a medical practitioner or a medical board.

27. The method according to claim 22, additionally comprising the step of performing statistical analysis for clinical investigation of diagnostic and treatment protocols.

28. The method according to claim 22, additionally comprising the step of performing statistical analysis for improving the diagnostic and treatment protocols with or without the aid of a medical practitioner or a medical board.

29. The method according to claim 22, additionally comprising the step of analyzing the collected data according to widely accepted clinical investigation standards to augment the reliability of treatment and diagnostic protocols.

30. The method according to claim 22, additionally comprising the step of collecting therapy data for future review and/or for statistic analysis.

31. The method according to claim 22 or any of its dependent claims, provided by a healthcare provider (i.e., Enterprise) system comprising a server computer; at least one end unit; an on-line connection to the sub-systems; an on-line connection to the main system, and management software.
